(19) United States
(12) Patent Application Publication
(10) Pub. No.: US 2025/0221913 A1
(43) Pub. Date: Jul. 10, 2025

(54) COMPOSITION COMPRISING CERAMIDE AND MONO-RHAMNOLIPID

(71) Applicant: Evonik Operations GmbH, Essen (DE)

(72) Inventors: Stefan Julian Liebig, Duesseldorf (DE); Nadine Truchan, Duesseldorf (DE); Svenja Blaschek, Gelsenkirchen (DE); Hacer Yalcinkaya-Demirel, Essen (DE); Kristin Klostermann, Essen (DE); Cornelia Pyko, Bad Vilbel (DE); Monica Desiree van Logchem, PA Zevenbergen (NL); Benjamin Tuchscherer, Dortmund (DE); Ursula Maczkiewitz, Essen (DE)

(73) Assignee: Evonik Operations GmbH, Essen (DE)

(21) Appl. No.: 18/978,374

(22) Filed: Dec. 12, 2024

(30) Foreign Application Priority Data
Jan. 5, 2024 (EP) 24150530.4

(51) Int. Cl. A61K 8/68; A61K 8/60; A61Q 5/00; A61Q 5/02; A61Q 5/12

(52) U.S. Cl. CPC A61K 8/68; A61K 8/60; A61Q 5/002; A61Q 5/006; A61Q 5/02; A61Q 5/12; A61K 2800/262; A61K 2800/5922; A61K 2800/805

(57) ABSTRACT

A composition has at least one ceramide and at least one mono-rhamnolipid. The composition may have a clear appearance. A process may involve producing a cosmetic or pharmaceutical formulation having at least one ceramide, by providing the aforementioned composition and adding a cosmetic or pharmaceutical acceptable carrier. A mono-rhamnolipid may be used for solubilizing and/or physical stabilization of a ceramide-containing composition.

### CROSS-REFERENCE TO RELATED APPLICATIONS

This patent application claims priority to European Patent Application No. 24150530.4, filed on Jan. 5, 2024, in the European Patent Office, the entire disclosure of which is hereby incorporated by reference herein.

### BACKGROUND OF THE INVENTION

### Field of the Invention

The invention relates to a composition comprising ceramide and mono-rhamnolipid.

### Description of Related Art

Ceramides are a class of lipophilic amides which form the lipid matrix of the skin together with fatty acids and cholesterol. These Amides play an important role in the cosmetics and are established ingredients in various formulations for beneficial effects on skin barrier, trans epidermal water loss and further functions. Many formulations are based on emulsions with higher oil content. Only a few examples of water-based formulations are known as ceramides are hardly soluble substances in aqueous environments.

WO2023161179 discloses liposomal composition comprising biosurfactants and the use of the liposomal composition for encapsulation of at least one cosmetic, pharmaceutical and/or nutraceutical active ingredient. With this it provides liposomal compositions which allows solubilization of hardly soluble substances with outstanding long-time stability in terms of re-precipitation.

KR20190080060 discloses transparent formulations containing ceramides. One disadvantage of these formulation is the usage of ethoxylated surfactants. Another disadvantage is the need for an oily compound, which might be not desired in an aqueous application.

U.S. Pat. No. 8,313,755B2 discloses clear aqueous ceramide compositions with polyhydric alcohols.

### SUMMARY OF THE INVENTION

It is an object of the instant invention to provide compositions comprising ceramides with a clear appearance.

### DETAILED DESCRIPTION OF THE INVENTION

It was found, surprisingly, that a combination of mono-rhamnolipids and ceramides give clear aqueous compositions.

The present invention therefore provides compositions comprising mono-rhamnolipids and ceramides.

One advantage of the compositions according to the invention is their enhanced deposition of ceramides on the skin.

A further advantage is the improved penetration of the ceramides in the presence of mono-rhamnolipids.

Another advantage is that the compatibility with organic acids is very high.

Another advantage is the improved foam quality of the compositions according to the invention.

A further advantage is the foam boosting effect of the compositions according to the invention.

Another advantage is the good compatibility of the compositions according to the invention with other ingredients.

A further advantage is the enhanced skin barrier protection.

A further advantage is the enhanced deposition of ceramides on the hair in the composition according to the invention.

A further advantage is the improved skin feel of the composition according to the invention.

A further advantage is the improved conditioning effect on hair by the composition according to the invention.

A further advantage is the improved thickenability of formulations.

A further advantage is that the composition according to the invention can be easily incorporated into different formulations.

Another advantage is that the composition according to the invention can be obtained without any heating step.

Another advantage is the high stability of the composition according to the invention.

A further advantage is the mildness of the composition according to the invention.

The present invention therefore provides a composition comprising
A) at least one ceramide, and
B) at least one rhamnolipid,
characterized in that component B) comprises at least 50 wt.-% mono-rhamnolipids, wherein the weight percentages refer to all rhamnolipids comprised in the total composition.

In the context of the present invention, the term “ceramide” is understood to mean acylated sphingoid bases, where the sphingoid bases are preferably selected from sphingosine, sphinganine, 6-hydroxysphingosine and phytosphingosine, also in glycosylated form, for example as glucosylceramides.

When determining the amount of rhamnolipid only the mass of the rhamnolipid is taken into account while disregarding the counter ion of the salt, in case the rhamnolipid is present as a salt.

Where average values are stated hereinbelow, then, unless stated otherwise, these are number-averaged average values.

Unless stated otherwise, percentages are data in percent by weight. The same is true for parts per million (ppm).

Wherever measurement values are stated hereinbelow, then, unless stated otherwise, these have been determined at a temperature of 25° C. and a pressure of 1013 mbar.

A preferred composition according to the instant invention is characterized in that said at least one ceramide is selected from the group comprising, preferably consisting of, ceramide NP, ceramide AP, ceramide EOP, ceramide NG (also known as ceramide NDS and ceramide 2), ceramide ADS, ceramide EODS, ceramide NS, ceramide AS, ceramide EOS, ceramide NH, ceramide AH and ceramide EOH, preferably selected from the group comprising ceramide NP, ceramide NG, ceramide AP and ceramide EOP, most preferably ceramide NP.

A preferred composition according to the instant invention is characterized in that component A) is comprised in an amount of from 0.0005 wt.-% to 5.0 wt.-%, more preferably from 0.001 wt.-% to 2.0 wt.-%, more preferably from 0.002 wt.-% to 1.5 wt.-%, even more preferably from 0.05 wt.-% to 1.0 wt.-%, where the percentages by weight refer to the total composition.

A preferred composition according to the invention is characterized in that said composition comprises at least two ceramides, preferably at least three ceramides, particularly preferably precisely three ceramides.

The term “rhamnolipids” in the context of the present invention preferably is understood to mean particularly compounds of the general formula (I) and salts thereof,

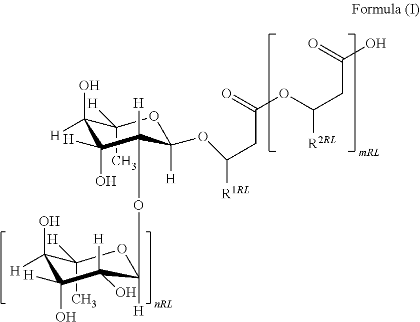

where
mRL=2, 1 or 0, preferably 1 or 0,
nRL=1 or 0,
R^{1RL} and R^{2RL}=mutually independently, identical or different, organic residues having 2 to 24, preferably 5 to 13 carbon atoms, in particular optionally branched, optionally substituted, particularly hydroxy-substituted, optionally unsaturated, in particular optionally mono-, bi- or tri-unsaturated alkyl residues, preferably those selected from the group consisting of pentenyl, heptenyl, nonenyl, undecenyl and tridecenyl and (CH₂)ₒ—CH₃ where o=1 to 23, preferably 4 to 12. If nRL=1, the glycosidic bond between the two rhamnose units is preferably in the α-configuration. The optically active carbon atoms of the fatty acids are preferably present as R-enantiomers (e.g. (R)-3-{(R)-3-[2-O-(α-L-rhamnopyranosyl)-α-L-rhamnopyranosyl]oxydecanoyl}oxydecanoate).

The term “di-rhamnolipid” in the context of the present invention is understood to mean compounds of the general formula (I) or salts thereof, where nRL=1.

The term “mono-rhamnolipid” in the context of the present invention is understood to mean compounds of the general formula (I) or salts thereof, where nRL=0.

Distinct rhamnolipids are abbreviated according to the following nomenclature: “diRL-CXCY” are understood to mean di-rhamnolipids of the general formula (I), in which one of the residues R^{1RL} and R^{2RL}=(CH₂)ₒ—CH₃ where o=X-4 and the remaining residue R¹ or R²=(CH₂)ₒ—CH₃ where o=Y-4.

“monoRL-CXCY” are understood to mean mono-rhamnolipids of the general formula (I), in which one of the residues R^{1RL} and R^{2RL}=(CH₂)ₒ—CH₃ where o=X-4 and the remaining residue R^{1RL} or R^{2RL}=(CH₂)ₒ—CH₃ where o=Y-4.

The nomenclature used therefore does not distinguish between “CXCY” and “CYCX”.

For rhamnolipids where mRL=0, monoRL-CX or diRL-CX is used accordingly.

If one of the abovementioned indices X and/or Y is provided with “:Z”, this signifies that the respective residue R^{1RL} and/or R^{2RL} is equal to an unbranched, unsubstituted hydrocarbon residue having X-3 or Y-3 carbon atoms having Z double bonds.

The following listed rhamnolipids, given that they comprise di-rhamnolipids, can be adjusted to the desired high content of mono-rhamnolipids by for example rhamnosidases.

Rhamnolipids applicable in the context of the instant invention can also be produced by fermentation of Pseudomonas, especially Pseudomonas aeruginosa, which are preferably non genetically modified cells, a technology already disclosed in the eighties, as documented e.g. in EP0282942 and DE4127908. Rhamnolipids produced in Pseudomonas aeruginosa cells which have been improved for higher rhamnolipid titres by genetical modification can also be used in the context of the instant invention; such cells have for example been disclosed by Lei et al. in Biotechnol Lett. 2020 June; 42(6):997-1002.

Rhamnolipids produced by Pseudomonas aeruginosa are commercially available from Jeneil Biotech Inc., e.g. under the tradename Zonix, from Logos Technologies (technology acquired by Stepan), e.g. under the tradename NatSurFact, from Biotensidion GmbH, e.g. under the tradename Rhapynal, from AGAE technologies, e.g. under the name R90, R95, R95Md, R95Dd, from Locus Bio-Energy Solutions and from Shanghai Yusheng Industry Co. Ltd., e.g. under the tradename Bio-201 Glycolipids.

The composition according to the instant invention is preferably characterized in that component B) comprises
12 wt.-% to 32 wt.-% monoRL-C8C10,
51 wt.-% to 81 wt.-% monoRL-C10C10,
1 wt.-% to 9 wt.-% monoRL-C10C12,
1 wt.-% to 9 wt.-% monoRL-C10C12:1,
wherein the weight percentages refer to all mono-rhamnolipids comprised in the composition.

A preferred composition according to the instant invention is characterized in that it is a non-liposomal composition.

The term “non-liposomal composition” means, that the composition does not comprise liposomes.

A preferred composition according to the instant invention is characterized in that it is does not comprise phospholipids.

A preferred composition according to the instant invention is characterized in that it comprises particles with a mean particle size of 13 nm or less, preferably in the range of from 3 nm to 12 nm. Photon correlation spectroscopy is employed in order to determine the mean particle size preferably at a total concentration of component A) of 0.5 wt. % with the composition comprising water. The measurement is performed using a Zetasizer Nano ZS90, Malvern Instruments Ltd., UK, according to the manufacturer's instruction. The Z-average is the intensity weighted mean hydrodynamic size of the ensemble collection of particles measured by dynamic light scattering (DLS). The Z-average is derived from a Cumulants analysis of the measured correlation curve, wherein a single particle size is assumed and a single exponential fit is applied to the autocorrelation function (see Zetasizer Nano ZS90 User Manual MAN0485-1-1 9 Jun. 2017).

A preferred composition according to the instant invention is characterized in that component B) is comprised in an amount of from 0.1 wt.-% to 15.0 wt.-%, preferably from 0.5 wt.-% to 10.0 wt.-%, more preferably from 1.0 wt.-% to 8.0 wt.-%, wherein the percentages by weight refer to the total composition.

According to the invention, the ratio by weight of component A) to component B) in the composition according to the invention is preferably from 0.0005:15 to 0.1:1, preferably from 0.005:10 to 0.5:5, particularly preferably from 0.05:7 to 0.1:5.

A preferred composition according to the instant invention is characterized in that it comprises
C) cholesterol and/or at least one cholesterol derivative selected from the group comprising cholesterol sulfate, cholesterol hydrogen succinate and 7-dehydrocholesterol.

Component c) preferably is comprised in an amount of from 0.01 wt.-% to 3.0 wt.-%, preferably from 0.02 wt.-% to 2.0 wt.-%, more preferably from 1.0 wt.-% to 0.05 wt.-%, where the percentages by weight refer to the total composition.

A preferred composition according to the instant invention is characterized in that it comprises
D) at least one sphingoid base, preferably selected from the group comprising sphingosine, sphinganine, 6-hydroxysphingosine, N-acetylphytosphingosine and phytosphingosine, especially phytosphingosine.

Component D) preferably is comprised in an amount of from 0.001 wt.-% to 2.0 wt.-%, preferably from 0.002 wt.-% to 1.5 wt.-%, more preferably from 0.05 wt.-% to 1.0 wt.-%, where the percentages by weight refer to the total composition.

A preferred composition according to the instant invention is characterized in that it comprises instead of or additionally to component D)
E) at least one fatty acid, preferably selected from the group of fatty acids comprising 12 to 28 carbon atoms, especially palmitic acid, stearic acid, octadecanoic acid, arachidic acid, behenic acid, docosanoic acid and lignoceric acid.

Component E) preferably is comprised in an amount of from 0.01 wt.-% to 3.0 wt.-%, preferably from 0.02 wt.-% to 2.0 wt.-%, more preferably from 1.0 wt.-% to 0.05 wt.-%, where the percentages by weight refer to the total composition.

A preferred composition according to the instant invention is characterized in that it has a pH in the range of 4.0 to 8.0, preferably 4.5 to 7.4 particularly preferably 5.0 to 7.2.

The “pH” in connection with the present invention is defined as the value which is measured for the relevant composition at 22° C. after stirring for five minutes using a pH electrode calibrated in accordance with ISO 4319 (1977).

A preferred composition according to the instant invention is characterized in that it comprises water, preferably in the range of 75.0 wt.-% to 99.5 wt.-%, preferably from 80.0 wt.-% to 98.5 wt.-%, more preferably from 85.0 wt.-% to 98.0 wt.-%, based on the total composition.

The present invention further relates to a process for producing a cosmetic or pharmaceutical formulation comprising at least one ceramide, comprising the process steps of:
a) providing a composition according to the instant invention;
b) adding a cosmetic or pharmaceutical acceptable carrier, preferable water, and preferably one further cosmetical and pharmaceutical formulation ingredient.

Said one further cosmetical and pharmaceutical formulation ingredient of step b) is, of course different from any component A) to E) comprised in the composition of the instant invention.

Preferred compositions according to the instant invention are preferably used in the context of the process according to the instant invention.

Said further cosmetical and pharmaceutical formulation ingredient preferably added in step b) of the process of the instant invention is preferably selected from the group of surfactants, emollients, emulsifiers, thickeners, UV light protection filters, antioxidants, hydrotropes, solids and fillers, film formers, pearlescence additives, opacifiers, deodorant and antiperspirant active ingredients, insect repellents, self-tanning agents, preservatives, conditioning agents, perfumes, dyes, odour absorbers, superfatting agents and solvents, preferably perfumes, conditioning agents and thickeners,

A preferred process according to the instant invention is characterized in that said process comprises
c) solubilization of the formulation.

In consequence, cosmetic or pharmaceutical formulation produced by the process according to the instant invention preferably is a water based surfactant formulation.

To facilitate the solubilization step c) of the process according to the instant invention it is preferred in accordance with the invention that the at least one additional surfactant is present in process step c).

Said at least one additional surfactant present in process step c) of the instant invention is preferably selected from the group of anionic, cationic, non-ionic, semi-polar, amphoteric and zwitterionic surfactants.

The non-ionic surfactants used are preferably alkoxylated, advantageously ethoxylated, in particular primary alcohols having preferably 8 to 18 carbon atoms and on average 1 to 12 mol of ethylene oxide (EO) per mol of alcohol, in which the alcohol radical can be linear or preferably 2-position methyl-branched or can contain linear and methyl-branched radicals in a mixture, as are customarily present in oxo alcohol radicals. In particular, however, alcohol ethoxylates with linear radicals from alcohols of native origin having 12 to 18 carbon atoms, for example from coconut, palm, tallow fat or oleyl alcohol, and on average 2 to 8 EO per mol of alcohol are preferred. The preferred ethoxylated alcohols include, for example, C12-C14-alcohols with 3 EO, 4 EO or 7 EO, C9-C11-alcohol with 7 EO, C13-C15-alcohols with 3 EO, 5 EO, 7 EO or 8 EO, -9-C12-C18-alcohols with 3 EO, 5 EO or 7 EO and mixtures of these, such as mixtures of C12-C14-alcohol with 3 EO and C12-C18-alcohol with 7 EO. The stated degrees of ethoxylation are statistical average values which can be an integer or a fraction for a specific product. Preferred alcohol ethoxylates have a narrowed homolog distribution.

In addition to these non-ionic surfactants, it is also possible to use fatty alcohols with more than 12 EO. Examples thereof are tallow fatty alcohol with 14 EO, 25 EO, or 40 EO. Non-ionic surfactants which contain EO and PO (propylene oxide) groups together in the molecule can also be used. In this connection, it is possible to use block copolymers with EO-PO block units or PO-EO block units, but also EO-PO-EO copolymers or PO-EO-PO copolymers.

It is of course also possible to use mixed alkoxylated non-ionic surfactants in which EO and PO units are not distributed blockwise, but randomly. Such products are obtainable as a result of the simultaneous action of ethylene oxide and propylene oxide on fatty alcohols.

Furthermore, alkyl glycosides can also be used as further non-ionic surfactants.

A further class of preferably used non-ionic surfactants, which are used either as the sole non-ionic surfactant or in combination with other non-ionic surfactants, are alkoxylated, preferably ethoxylated or ethoxylated and propoxylated fatty acid alkyl esters, preferably having 1 to 4 carbon atoms in the alkyl chain, in particular fatty acid methyl esters, as are described for example in the Japanese patent application JP 58/217598 or which are preferably prepared by the process described in the international patent application WO-A-90/13533.

Non-ionic surfactants of the amine oxide type, for example N-cocoalkyl-N,N-dimethylamine oxide and N-tallowalkyl-N,N-dihydroxyethylamine oxide, and of the fatty acid alkanolamide type may also be suitable. The amount of these non-ionic surfactants is preferably not more than that of the ethoxylated fatty alcohols, in particular not more than half thereof.

Further suitable surfactants are polyhydroxy fatty acid amides; the polyhydroxy fatty acid amides are substances which can usually be obtained by reductive amination of a reducing sugar with ammonia, an alkylamine or an alkanolamine and subsequent acylation with a fatty acid, a fatty acid alkyl ester or a fatty acid chloride.

Further suitable non-ionic surfactants are polyglycerol partial esters based on mono- and dicarboxylic acids and crosslinked polyglycerol partial esters based on mono- and dicarboxylic acids.

The anionic surfactants used are, for example, those of the sulphonate and sulphate type. Suitable surfactants of the sulphonate type here are preferably C9-C13-alkylbenzenesulphonates, olefinsulphonates, i.e. mixtures of alkene- and hydroxyalkanesulphonates, and also disulphonates, as are obtained, for example, from C12-C18-monoolefins with a terminal or internal double bond by sulphonation with-10-gaseous sulphur trioxide and subsequent alkaline or acidic hydrolysis of the sulphonation products. Also of suitability are alkanesulphonates which are obtained from C12-C18-alkanes, for example by sulphochlorination or sulphoxidation with subsequent hydrolysis or neutralization. Similarly, the esters of a-sulpho fatty acids (ester sulphonates), for example the a-sulphonated methyl esters of hydrogenated coconut, palm kernel or tallow fatty acids, are also suitable.

Further suitable anionic surfactants are sulphated fatty acid glycerol esters. Fatty acid glycerol esters are to be understood as meaning the mono-, di- and triesters, and also mixtures thereof, as are obtained in the preparation by esterification of a monoglycerol with 1 to 3 mol of fatty acid or in the transesterification of triglycerides with 0.3 to 2 mol of glycerol. Preferred sulphated fatty acid glycerol esters here are the sulphation products of saturated fatty acids having 6 to 22 carbon atoms, for example of caproic acid, caprylic acid, capric acid, myristic acid, lauric acid, palmitic acid, stearic acid or behenic acid.

Preferred alk(en)yl sulphates are the alkali metal and in particular the sodium salts of the sulphuric acid half-esters of the C12-C18-fatty alcohols, for example from coconut fatty alcohol, tallow fatty alcohol, lauryl, myristyl, cetyl or stearyl alcohol or the C10-C20-oxo alcohols and those half-esters of secondary alcohols of these chain lengths. Furthermore, preference is given to alk(en)yl sulphates of the specified chain length which contain a synthetic straight-chain alkyl radical prepared on a petrochemical basis, and which have an analogous degradation behaviour to the suitable compounds based on fatty chemical raw materials.

The sulphuric acid monoesters of the straight-chain or branched C7-C20-alcohols ethoxylated with 1 to 6 mol of ethylene oxide, such as 2-methyl-branched C9-alcohols having on average 3.5 mol of ethylene oxide (EO) or C12-C18-fatty alcohols with 1 to 4 EO, are also suitable. On account of their high foaming behaviour, they are used in cleaning compositions only in relatively small amounts, for example in amounts of from 1 to 5% by weight.

Further suitable anionic surfactants are also the salts of alkylsulphosuccinic acid, which are also referred to as sulphosuccinates or as sulphosuccinic acid esters and constitute the monoesters and/or diesters of sulphosuccinic acid with alcohols, preferably fatty-11-alcohols and in particular ethoxylated fatty alcohols. Preferred sulphosuccinates contain C8-C18-fatty alcohol radicals or mixtures of these. Particularly preferred sulphosuccinates contain a fatty alcohol radical which is derived from ethoxylated fatty alcohols. In this connection, sulphosuccinates whose fatty alcohol radicals are derived from ethoxylated fatty alcohols with a narrow homolog distribution are particularly preferred in turn. It is likewise also possible to use alk(en)ylsuccinic acid having preferably 8 to 18 carbon atoms in the alk(en)yl chain or salts thereof.

Particularly preferred anionic surfactants are soaps. Also of suitability are saturated and unsaturated fatty acid soaps, such as the salts of lauric acid, myristic acid, palmitic acid, stearic acid, (hydrogenated) erucic acid and behenic acid, and also soap mixtures derived in particular from natural fatty acids, for example coconut, palm kernel, olive oil or tallow fatty acid.

The anionic surfactants including the soaps can be in the form of their sodium, potassium or ammonium salts, as well as soluble salts of organic bases, such as mono-, di- or triethanolamine. Preferably, the anionic surfactants are in the form of their sodium or potassium salts, in particular in the form of the sodium salts.

Amphoteric surfactants which can be used according to the invention are those surface-active compounds which carry at least one quaternary ammonium group and at least one —CO₂— or —SO₃— group in the molecule. Particularly preferred amphoteric surfactants in this connection are betaine surfactants such as alkyl- or alkylamidopropylbetaines. In particular, betaines such as the N-alkyl-N,N-dimethylammonium glycinates, e.g. the cocoalkyldimethylammonium glycinate, N-acylaminopropyl-N, N-dimethylammonium glycinates, e.g. the cocoacylaminopropyldimethylammonium glycinate, the C12-C18-alkyldimethylacetobetaine, the cocoamidopropyldimethylacetobetaine, 2-alkyl-3-carboxymethyl-3-hydroxyethylimidazolines and sulphobetaines having in each case 8 to 18 carbon atoms in the alkyl or acyl group, and also the cocoacylaminoethylhydroxyethylcarboxymethyl glycinate are preferred here. A particularly preferred zwitterionic surfactant is the N,N-dimethyl-N-(lauroylamidopropyl)ammoniumacetobetaine known under the INCI name Cocamidopropyl Betaine.

Further suitable amphoteric surfactants are formed by the group of amphoacetates and amphodiacetates, in particular, for example, coco- or laurylamphoacetates or -diacetates, the group of amphopropionates and amphodipropionates, and the group of amino acid-based surfactants such as acyl glutamates, in particular disodium cocoyl-12-glutamate and sodium cocoyl glutamate, acyl glycinates, in particular cocoyl glycinates, and acyl sarcosinates, in particular ammonium lauroyl sarcosinate and sodium cocoyl sarcosinate.

Said at least one additional surfactant present in process step c) of the instant invention is preferably selected from biosurfactants different from rhamnolipids, preferably glycolipids, more preferably selected from sophorolipids and glucolipids.

A preferred process according to the instant invention is characterized in that said process comprises
d) adjusting the pH of the formulation to a range of 4.0 to 8.0, preferably 4.5 to 7.4 particularly preferably 5.0 to 7.2.

The formulations prepared by the process according to the invention are in particular physically stable formulations. In the context of the present invention, the term “physically stable formulations containing at least one ceramide” is understood in particular to mean formulations which in particular do not exhibit any crystallization of the ceramides and the sphingoid base and no separation or inhomogeneity after six months of storage at 25° C.

The present invention further relates to the use of mono-rhamnolipid, preferably in an amount of from 0.1 wt.-% to 15.0 wt.-%, preferably from 0.5 wt.-% to 10.0 wt.-%, more preferably from 1.0 wt.-% to 8.0 wt.-%, wherein the percentages refer to the total composition, for solubilizing and/or physical stabilization of a ceramide-containing composition, in particular with regard to homogeneity, and/or for preventing crystallization of at least one ceramide in a composition.

In the context of the use according to the instant invent the ceramide-containing composition are preferably equal to the preferred compositions according to the instant invention, especially regarding components A) and B).

The examples adduced hereinafter describe the present invention by way of example, without any intention that the invention, the scope of application of which is apparent from the entirety of the description, be restricted to the embodiments specified in the examples.

### EXAMPLES

### Example 1: Preparation of Highly Concentrated Mono-Rhamnolipids

Mono-rhamnolipids were produced by a fermentation of a Pseudomonas putida strain pBBR1MCS2-Plac-rhlAB, which was created just like the Pseudomonas putida strain pBBR1MCS2-Plac-rhlABC-T-Ptac-rhlC-T described in EP2786743, yet the Bsu361 restriction site was inserted directly behind the stop codon of the rhlB gene, thereby omitting rhlC. The preculture in a shake flask was carried out as described in EP2598646. For the main culture, a mineral medium (M9) was likewise employed. The fermentation was conducted in a 2-litre fermenter in a carbon-limited manner via a glucose feed input. The glucose feed input takes place by reference to the dissolved oxygen signal. The dissolved oxygen was regulated at 20% saturation via the stirrer speed. The pH is regulated to 7 via a pH electrode and addition of 2M sulphuric acid or a 20% by weight ammonia solution. To prevent excessive foaming of the fermentation broth, the defoamer Dow Corning 1500 was added as required. The fermentation was conducted over 4 days to a dry biomass of 16 g/L. The mono-rhamnolipid concentration was determined by HPLC and was 8.5 g/L.

After separating off the cells by means of centrifugation at 10,000 g, the fermentation broth was adjusted to a pH of 3.1 by adding concentrated H₂SO₄.

A multiphase composition was obtained, which was separated by centrifugation at 10,000 g and the upper aqueous phase was discarded.

The remnant was treated further.

The compositions listed in table1 were obtained by elevating the pH using KOH (aq) and diluting with water to the given mono-rhamnolipid concentration; percentage by weight referring to the total composition.

### Example 2: Preparation of Clear Aqueous Solutions of Ceramides

The dissolution capacity of the mono-rhamnolipid was investigated by mixing these with ceramides. Ceramides were added to a mono-rhamnolipid solution and heating to a temperature of 50° C. and afterwards water is added in the ratios given in table 1 and the pH was adjusted to the given value. The solutions were cooled to room temperature and optical evaluation was performed directly and 24 h after preparation of the solutions.

The evaluation of the maximum amount ceramide solubilized is determined by the mixture resulting in a clear formulation. The sample was assessed visually and additionally by microscopic analysis using polarized light. When no illumination of the sample is observed the solution is free from crystals and according to the definition clear.

As shown in table 1 surprisingly the combinations of mono-rhamnolipids and ceramides gave clear solutions, whereas combination of ceramides and di-rhamnolipid or PEG-40 Hydrogenated Castor Oil resulted in turbid solutions.

TABLE 1
Examples of combinations of mono-rhamnolipids, di-rhamnolipids,
PEG-40 Hydrogenated Castor Oil and ceramides

| | Example 2a | Example 2b | Example 2c | Example 2d | Example 2e |
|---|---|---|---|---|---|
| | according to | according to | according to | not according | not according |
| | the invention | the invention | the invention | to the invention | to the invention |
| Appearance | Clear solution | Clear solution | Clear solution | Turbid solution | Turbid solution |
| Appearance with | No crystals | No crystals | No crystals | Turbid Solution | Turbid Solution |
| polarized light | visible | visible | visible | | |
| Content mono-RL | 3.7 | 3.7 | 3.9 | | |
| active in % | | | | | |
| Content di-RL active in | | | | 3.9 | |
| % | | | | | |
| Content PEG-40 | | | | | 3.9 |
| Hydrogenated Castor | | | | | |
| Oil in % | | | | | |
| Content Ceramide | 0.15 | 0.20 | 0.19 | 0.19 | 0.19 |
| NP(Ceramide 3B) in % | | | | | |
| Content Water in % | ad 100 | ad 100 | ad 100 | ad 100 | ad 100 |
| pH | 7 | 7 | 7 | 7 | 7 |

### Example 3

The dissolution capacity of the mono-rhamnolipid was investigated by mixing these with ceramides. Ceramides were added to a mono-rhamnolipid solution and heating to a temperature of 70° C. and afterwards water is added in the ratios given in table 1 and the pH was adjusted to the given value. The solutions were cooled to room temperature and optical evaluation was performed directly and 24 h after preparation of the solutions.

The evaluation of the maximum amount ceramide solubilized is determined by the mixture resulting in a clear formulation. The sample was assessed visually and additionally by microscopic analysis using polarized light. When no illumination of the sample is observed the solution is free from crystals and according to the definition clear.

As shown in table 2 surprisingly the combinations of mono-rhamnolipids and ceramides gave clear solutions, whereas combination of ceramides and di-rhamnolipid or PEG-40 Hydrogenated Castor Oil resulted in turbid solutions.

TABLE 2
Examples of combinations of mono-rhamnolipids, di-rhamnolipids,
PEG-40 Hydrogenated Castor Oil and ceramides

| | Example 3a | Example 3b | Example 3c | Example 3d | Example 3e |
|---|---|---|---|---|---|
| | according to | according to | according to | not according | not according |
| | the invention | the invention | the invention | to the invention | to the invention |
| Appearance | Clear solution | Clear solution | Clear solution | Turbid solution | Turbid solution |
| Appearance with | No crystals | No crystals | No crystals | Turbid Solution | Turbid Solution |
| polarized light | visible | visible | visible | | |
| Content mono-RL | 3.7 | 3.7 | 3.9 | | |
| active in % | | | | | |
| Content di-RL active in | | | | 3.9 | |
| % | | | | | |
| Content PEG-40 | | | | | 3.9 |
| Hydrogenated Castor | | | | | |
| Oil in % | | | | | |
| Content Ceramide NG | 0.15 | 0.20 | 0.19 | 0.19 | 0.19 |
| in % | | | | | |
| Content Water in % | ad 100 | ad 100 | ad 100 | ad 100 | ad 100 |
| pH | 7 | 7 | 7 | 7 | 7 |

### Example Formulations

In the below “Composition A” refers to the following two compositions A1 and A2 given figures in wt.-%:

| | Composition | Composition |
|---|---|---|
| | A1 | A2 |
| L-(+)-lactic acid | 3 | 3 |
| Ceramide NP | 3 | — |
| Ceramide NG | — | 3 |
| mono-Rhamnolipid from example 1 | 75 | 75 |
| Water | ad 100 | ad 100 |
| | pH 3 | pH 3 |

Thus, each formulations listed below is disclosed in two versions.

### Example Formulation 1: Volume and Body Shampoo

| Ingredient | % |
|---|---|
| Aqua | ad 100 |
| Sodium Lauryl Sulfate | 7.0 |
| Sodium Laureth Sulfate | 5.0 |
| Cocamidopropyl Betaine | 2.0 |
| Composition A | 2.0 |
| Sodium Citrate | 1.5 |
| Sodium Xylenesulfonate | 1.5 |
| Sodium Chloride | 1.4 |
| Citric Acid | ad pH 5.5 |
| Sodium Levulinate, Sodium Benzoate | 1.0 |
| Hydroxypropyl Methylcellulose | 1.0 |
| Tetrasodium EDTA | 0.8 |
| Butylphenyl Methylpropional | 0.5 |
| Panthenol | 0.5 |
| Panthenyl Ethyl Ether | 0.3 |
| Starch Hydroxypropltrimonium chloride | 0.2 |
| Linalool | 0.1 |
| Hexyl Cinnamal | 0.1 |
| Limonene | 0.1 |
| Benzyl Salicylate | 0.2 |
| Magnesium Nitrate | 0.1 |
| Magnesium Chloride | 0.1 |
| Methylchloroisothiazolinone | 0.1 |
| Dyes | q.s. |

### Example Formulation 2: Repair Shampoo

| Ingredient | % |
|---|---|
| Aqua | ad 100 |
| Sodium Laureth Sulfate | 7.0 |
| Glycolipids | 3.0 |
| Composition A | 3.0 |
| Glycol Distearate | 2.5 |
| Cocamidopropyl Betaine | 2.0 |
| Sodium Citrate | 1.8 |
| Cocamide MEA | 1.8 |
| Sodium Xylenesulfonate | 1.5 |
| Dimethicone | 1.5 |
| Citric Acid | ad pH 5.0 |
| Sodium Benzoate | 0.7 |
| Polyquaternium-76 | 0.5 |
| Sodium Chloride | 0.9 |
| Glycerin | 0.5 |
| Tetrasodium EDTA | 0.3 |
| Butylphenyl Methylpropional | 0.2 |
| Hexyl Cinnamal | 0.1 |
| Linalool | 0.1 |
| alpha-Isomethyl Ionone | 0.1 |
| Geraniol | 0.2 |
| Hydrochloric Acid | 0.1 |
| Magnesium Nitrate | 0.1 |
| Methylchloroisothiazolinone | 0.1 |
| Magnesium Chloride | 0.1 |
| Methylisothiazolinone | 0.1 |
| Dyes | q.s. |

### Example Formulation 3: Anti-Dandruff Shampoo

| Ingredient | % |
|---|---|
| Aqua | ad 100 |
| Sodium Laureth Sulfate | 6.0 |
| Sodium Lauryl Sulfate | 5.0 |
| Composition A | 4.0 |
| Cocamide MEA | 4.0 |
| Zinc Carbonate | 3.0 |
| Glycol Distearate | 2.5 |
| Dimethicone | 1.0 |
| Zinc Pyrithione | 1.0 |
| Sodium Chloride | 0.6 |
| Cetyl Alcohol | 0.8 |
| Guar Hydroxypropyltrimonium Chloride | 0.3 |
| Sodium Xylenesulfonate | 0.5 |
| Magnesium Sulfate | 0.7 |
| Sodium Benzoate | 0.7 |
| Ammonium Laureth Sulfate | 0.3 |
| Citric Acid | ad pH 4.5 |
| Benzyl Alcohol | 0.2 |
| Methylchloroisothiazolinone | 0.1 |
| Methylisothiazolinone | 0.1 |
| Parfum, Dyes | q.s. |

Example formulation 4: Strengthening Shampoo

| Ingredient | % |
|---|---|
| Water | ad 100 |
| Sodium Lauryl Sulfate | 6.0 |
| Sodium Laureth Sulfate | 4.0 |
| Cocamidopropyl Betaine | 3.0 |
| Composition A | 3.0 |
| Glycol Distearate | 2.5 |
| Dimethicone | 2.0 |
| Sodium Citrate | 1.5 |
| Cocamide MEA | 1.0 |
| Sodium Xylenesulfonate | 1.0 |
| Citric Acid | ad pH 5.0 |
| Sodium Benzoate | 0.7 |
| Sodium Chloride | 0.8 |
| Tetrasodium EDTA | 0.3 |
| Polyquaternium-6 | 0.5 |
| Honey (Mel) | 0.5 |
| Prunus Armeniaca (Apricot) Fruit Extract | 0.5 |
| Methylchloroisothiazolinone | 0.1 |
| Methylisothiazolinone | 0.1 |
| Parfum, Dyes | q.s. |

Example formulation 5: Shampoo

| Ingredient | % |
|---|---|
| Aqua | ad 100 |
| Sodium Laureth Sulfate | 9.0 |
| Cocamidopropyl Betaine | 3.0 |
| Composition A | 3.0 |
| Glycerin | 2.5 |
| Sodium Chloride | 1.7 |
| Glycol Distearate | 1.5 |
| Panthenol | 0.2 |
| Propylene Glycol | 0.3 |
| Hydroxypropyl Guar Hydroxypropyltrimonium Chloride | 0.3 |
| Sodium Benzoate | 0.7 |
| PEG-55 Propylene Glycol Oleate | 0.5 |
| Citric Acid | ad pH 5.5 |
| Hydrolyzed Wheat Protein | 0.3 |
| Argania Spinosa Kernel Oil | 0.1 |
| Laureth-4 | 0.5 |
| Potassium Sorbate | 0.2 |
| Parfum, Dyes | q.s. |

### Example Formulation 6: Moisturising Shampoo

| Ingredient | % |
|---|---|
| Aqua | ad 100 |
| Sodium Laureth Sulfate | 8.0 |
| Cocamidopropyl Betaine | 3.0 |
| Sodium Chloride | 2.2 |
| Composition A | 2.0 |
| Hydrolyzed Keratin | 2.0 |
| Water Lily (Nymphaea Odorata) Root Extract | 1.8 |
| Aloe Barbadensis Leaf Juice | 1.5 |
| Glyceryl Glucoside | 1.0 |
| Panthenol | 1.0 |
| Decyl Glucoside | 1.0 |
| Polyquaternium-10 | 0.2 |
| Guar Hydroxypropyltrimonium Chloride | 0.3 |
| PEG-40 Hydrogenated Castor Oil | 0.7 |
| Glycerin | 0.5 |
| Citric Acid | ad pH 5.0 |
| Sodium Benzoate | 1.2 |
| Propylene Glycol | 1.0 |
| Citronellol | 0.1 |
| Limonene | 0.1 |
| alpha-Isomethyl Ionone | 0.1 |
| Benzyl Alcohol | 0.2 |
| Dyes | q.s. |

1. A composition, comprising:
A) at least one ceramide, and
B) at least one rhamnolipid,
wherein B) comprises at least 50 wt.-% mono-rhamnolipids, wherein weight percentages refer to all rhamnolipids comprised in the total composition.

2. The composition according to claim 1, wherein said at least one ceramide is selected from the group consisting of ceramide NP, ceramide AP, ceramide EOP, ceramide NG, ceramide ADS, ceramide EODS, ceramide NS, ceramide AS, ceramide EOS, ceramide NH, ceramide AH and ceramide EOH.

3. The composition according to claim 1, having A) in an amount of from 0.0005 wt.-% to 5.0 wt.-%, where percentages by weight refer to the total composition.

4. The composition according to claim 1, wherein B) comprises:
12 wt.-% to 32 wt.-% monoRL-C8C10,
51 wt.-% to 81 wt.-% monoRL-C10C10,
1 wt.-% to 9 wt.-% monoRL-C10C12,
1 wt.-% to 9 wt.-% monoRL-C10C12:1,
wherein weight percentages refer to all mono-rhamnolipids in the composition.

5. The composition according to claim 1, having B) in an amount of from 0.1 wt.-% to 15.0 wt.-%, wherein the percentages by weight refer to the total composition.

6. The composition according to claim 1, further comprising:
C) cholesterol and/or at least one cholesterol derivative selected from the group consisting of cholesterol sulfate, cholesterol hydrogen succinate and 7-dehydrocholesterol.

7. The composition according to claim 1, further comprising:
D) at least one sphingoid base.

8. The composition according to claim 1, the composition further comprising:
E) at least one fatty acid.

9. The composition according to claim 1, wherein the composition has a pH in a range of 4.0 to 8.0.

10. The composition according to claim 1, further comprising:
water.

11. A process for producing a cosmetic or pharmaceutical formulation comprising at least one ceramide, the process comprising:
a) providing a composition according to claim 1; and
b) adding a cosmetic or pharmaceutical acceptable carrier.

12. The process according to claim 11, further comprising:
c) solubilization of the formulation.

13. The process according to claim 12, further comprising:
d) adjusting the pH of the formulation to a range of
4.0 to 8.0.

14. A composition for solubilizing and/or physical stabilization of a ceramide-containing composition, and/or for preventing crystallization of at least one ceramide in a composition, the composition comprising:
a mono-rhamnolipid.
